(19) United States
(12) Patent Application Publication
(10) Pub. No.: US 2011/0150873 A1
(43) Pub. Date: Jun. 23, 2011

(54) ANTI-INFLAMMATORY COMPOSITIONS AND COMBINATIONS

(75) Inventor: David John Grainger, Cambridge (GB)

(73) Assignee: CAMBRIDGE ENTERPRISE LIMITED, Cambridge (GB)

(21) Appl. No.: 12/747,878

(22) PCT Filed: Dec. 10, 2008
(86) PCT No.: PCT/GB2008/004074

§ 371 Date: Nov. 19, 2010

(30) Foreign Application Priority Data
Dec. 12, 2007 (GB) 0724277.9

(51) Int. Cl. A61K 39/395; A61K 31/55; A61K 31/4412; A61K 31/4015; A61K 31/573; A61K 31/616; A61K 31/405; A61K 31/4406; A61K 31/415; A61K 31/5415; A61K 31/542; A61K 31/454; C07D 223/12; C07D 211/88; C07D 207/273; C07D 209/28; C07D 513/04; C07D 279/02; C07D 213/76; C07D 231/12; C07K 19/00; C07K 16/24; C07D 401/04; A61P 29/00; A61P 37/06; A61P 9/00; A61P 19/10; A61P 35/00; A61P 25/28; A61P 17/06; A61P 17/00; A61P 11/06; A61P 11/00; A61P 1/00

(52) U.S. Cl. 424/134.1; 514/212.03; 514/349; 514/424; 514/171; 514/161; 514/420; 514/352; 514/406; 514/226.5; 424/133.1; 424/142.1; 514/323; 540/527; 546/297; 548/550; 548/500; 544/48; 544/49; 546/312; 548/375.1; 530/387.3; 530/388.15; 546/201

(57) ABSTRACT

The invention relates to the use of Broad-Spectrum Chemokine Inhibitors (BSCIs), and in particular members of the acylaminolactam class of pharmaceutical agents, for the prevention, prophylaxis, treatment or amelioration of symptoms of inflammatory diseases. In particular, improved compositions consisting of BSCI agents combined with one or more additional active pharmaceutical agents in order to achieve improved anti-inflammatory efficacy with a reduced side-effect profile are described and claimed.

The invention relates to the use of Broad-Spectrum Chemokine Inhibitors (BSCIs), and in particular members of the acylaminolactam class of pharmaceutical agents, for the prevention, prophylaxis, treatment or amelioration of symptoms of inflammatory diseases. In particular, improved compositions consisting of BSCI agents combined with one or more additional active pharmaceutical agents in order to achieve improved anti-inflammatory efficacy with a reduced side-effect profile are described and claimed.

Inflammation is an important component of physiological host defence. In response to various stimuli (such as infection or tissue damage) the immune system dispatches white blood cells (also known as leukocytes) to the affected area. These leukocytes then attack invading pathogens via a variety of mechanisms, including phagocytosis, release of toxic intermediates (such as superoxide radicals) and specific cell mediated killing. For mammals, including man, these defensive mechanisms are essential for survival. Pathological disruption of host defence (such as occurs following infection with the HIV virus) results in a vast array of opportunistic infections which are eventually lethal.

Increasingly, however, it is clear that temporally or spatially inappropriate inflammatory responses play a part in a wide range of diseases, including those with an obvious leukocyte component (such as autoimmune diseases, asthma or atherosclerosis) but also in diseases that have not traditionally been considered to involve leukocytes (such as osteoporosis or Alzheimer's disease). In these diseases leukocytes are recruited to tissues by inappropriate triggers (such as an autoimmune reaction, where antibodies inadvertently recognise a host protein, or accumulated tissue damage, such as persistant apoptotic bodies, extracellular cholesterol deposits or particulate matter in the lungs). Such diseases often become chronic because the recruited leukocytes are unable to deal with the trigger (they cannot, for example, remove or kill all host cells expressing an autoantigen or engulf particulates which are too large for the cell), and continually release pro-inflammatory cytokines which recruit further leukocytes to the vain task.

Treating the inflammatory component of such diseases has been a major goal of the global pharmaceutical industry for a number of decades, and a wide variety of useful treatments have been developed. Examples include the corticosteroids (a range of natural, semisynthetic and synthetic agents designed to mimic the effect of cortisol, including prednisolone, methylprednisolone, dexamethasone, betamethasone, fluticasone and so forth), cyclooxygenase inhibitors (both non-selective or cox-1 selective, such as indomethacin, sulfasalzine and aspirin, and more recently cox-2 selective, such as celecoxib), leukotriene blockers (such as monteleukast) and anti-TNFs (such as modified monoclonal neutralising antibodies, including infliximab (Remicade™) and adalimumab (Humira™), TNF receptor fusion proteins, such as etanercept (Enbrel™), as well as small molecule TNF-α synthesis inhibitors like thalidomide).

Unavoidably, however, such agents balance a beneficial effect on pathological inflammation with an undesirable immunosuppressive effect on host defence. In general, the stronger the anti-inflammatory effects of the medication, the greater the unintended immunosuppressive side-effects. Corticosteroids, for example, generally exhibit greater anti-inflammatory efficacy than other medicaments such as cyclooxygenase inhibitors, and are the first line therapy for many severe inflammatory conditions (such as asthma, psoriasis, eczema, irritable bowel syndrome and many others). However, this superior anti-inflammatory efficacy must be carefully weighed against the greater side-effect burden and dose and duration of treatment must be carefully monitored to achieve net benefit to the patient.

Side-effects from powerful anti-inflammatory medications, such as corticosteroids, are not limited to immunosuppression of host defence mechanisms (resulting in increased opportunistic infections, such as candidiasis, in patients receiving chronic, high dose steroid therapy). Cells of the immune system have been recruited into many processes not directly related to host defence: for example, specialised monocyte-derived cells such as osteoclasts play key roles in tissue homeostasis in a variety of tissues, such as bone. As a result, agents which interefere with immune cell function also have undesirable effects on such tissues. As a result, chronic corticosteroid therapy is associated with increased bone loss and eventually osteoporosis.

Corticosteroids mediate their effect through members of the nuclear hormone receptor family of proteins, which are intracellular receptors with ligand-dependent transcription factor activity. These receptors are not restricted to the cells of the immune system, and control important gene expression patterns in a host of tissues, including liver and pancreas. As a result, corticosteroid therapy also has side-effects associated with their action on non-immune cells. For example, in children chronic corticosteroid therapy (for the treatment of severe asthma, for instance) is associated with growth retardation as a result of suppressed growth hormone secretion from the pituitary. Similarly, chronic steroid therapy affects glucose homeostasis through interference with insulin and glucagon release from the pancreas, as well as disrupting electrolyte balance regulated by adrenal hormones such as aldosterone. These non-immune effects of steroid are collectively referred to as destabilisation of the HPA axis (an acronym for Hypothalamus, Pituitary and Adrenal axis, reflecting the interlinked signalling networks which link these three key endocrine organs). Perturbations of the HPA axis is usually the limiting factor on the dose and duration of steroid therapy, and significantly reduces the clinical utility of this otherwise highly effective class of anti-inflammatory medicaments.

Other, milder, anti-inflammatory medicaments are not, however, completely devoid of side-effects either. Although agents such as cyclooxygenase inhibitors, having less powerful effects on leukocyte function than steroids, do not have immunosuppressive effects on host defence (at least not to the extent that risk of acute infection is increased), they have unwanted effects mediated through non-immune cells. Non-selective, or cox-1 selective, cyclooxygenase inhibitors such as indomethacin, sulfasalazine or aspirin, have unwanted effects on the gut mucosa, and like steroids, it is side-effects that are the limiting factor for chronic use of these medicaments in diseases such as rheumatoid arthritis. Even newer, cox-2 selective cycloxygenase inhibitors, such as celecoxib, which have reduced gastrointestinal side-effects compared to earlier molecules, now appear to have off-target effects resulting in increased risk of heart attacks and other cardiovascular complications.

Since existing anti-inflammatory medications are generally considered to offer a trade-off between efficacy and side-effects, there have been many attempts to identify newer agents, with different molecular targets, which have greater selectivity for pathological inflammation, and hence less immunosuppressive effects on host defence or undesirable effects on non-immune cell types. One such approach has been to target chemokines.

The chemokines are a large family of signalling molecules with homology to interleukin-8, which have been implicated in regulating leukocyte trafficking both in physiological and pathological conditions. With more than fifty ligands and twenty receptors involved in chemokine signalling, the system has the requisite information density to address leukocytes through the complex immune regulatory processes from the bone marrow, to the periphery, then back through secondary lymphoid organs. However, this complexity of the chemokine system has at first hindered pharmacological approaches to modulating inflammatory responses through chemokine receptor blockade. It has proved difficult to determine which chemokine receptor(s) should be inhibited to produce therapeutic benefit in a given inflammatory disease.

More recently, a family of agents which block signalling by a wide range of chemokines simultaneously has been described: Reckless et al., Biochem J. (1999) 340:803-811. The first such agent, a peptide termed “Peptide 3”, was found to inhibit leukocyte migration induced by 5 different chemokines, while leaving migration in response to other chemoattractants (such as fMLP or TGF-beta) unaltered. This peptide, and its analogs such as NR58-3.14.3 (i.e. Sequence ID No.1 c(DCys-DGln-DIle-DTrp-DLys-DGln-DLys-DPro-DAsp-DLeu-DCys)-NH₂), are collectively termed “Broad Spectrum Chemokine Inhibitors” (BSCIs). Grainger et al., Biochem. Pharm. 65 (2003) 1027-1034 have subsequently shown BSCIs to have potentially useful anti-inflammatory activity in a range of animal models of diseases. Interestingly, simultaneous blockade of multiple chemokines is not apparently associated with acute or chronic toxicity, suggesting this approach may be a useful strategy for developing new anti-inflammatory medications with similar benefits to steroids but with reduced side-effects.

More recently, a range of small molecule BSCIs which are more suitable for use as human pharmaceuticals have been developed, including 16-amino and 16-aminoalkyl derivatives of the alkaloid yohimbine (Reference: Grainger et al., Mini Rev Med Chem 5 (2005) 825-32; WO 00/42071), as well as a range of N-substituted 3-aminoglutarimides (Reference: Fox et al., J Med Chem 45(2002) 360-370; WO 99/12968 and WO 00/42071) and N-substituted aminolactams (Reference: Fox et al., J Med Chem 48 (2005) 867-74 ; WO 05/053702).

One such family of stable, broad spectrum chemokine inhibitors (BSCIs) are the 3-amino caprolactams, with a seven-membered monolactam ring (see, for example, WO 05/053702 and WO 06/134385). However, further useful anti-inflammatory compounds have also been generated from other 3-aminolactams with different ring size (see for example WO 06/134385 and GB 07 15068.3). Other modifications to the lactam ring, including introduction of heteroatoms and bicyclolactam ring systems, also yield compounds with BSCI activity (see, for example, WO 06/018609 and WO 06/085096).

Previous disclosures have provided considerable information on selecting an appropriate BSCI for any particular application. For example, where high potency is required introduction of 2,2-disubstitution (at the alpha- or key-carbon atom in the acyl side chain of acyl-3-aminolactams) leads to a considerable increase in potency as a BSCI, both in vitro and in vivo in models of acute inflammation, whether the 2,2-disubstituted acyl group was open chain (see WO 05/053702), monocyclic (see WO 06/134384) or polycyclic (see WO 06/016152). Similarly, where excellent pharmacokinetic properties (resulting in higher exposures in vivo) are required, the compound 3-(2′,2′-dimethylpropanoylamino)-tetrahydropyridin-2-one was found to be particularly suitable (GB 07 15068.3).

BSCIs, like other agents intended for use as anti-inflammatory agents, will likely have side-effects, although to date the degree of anti-inflammatory efficacy which can be achieved for a given level of side-effects seems to be greater than for many other classes of agent. This likely reflects, at least in part, the ability of BSCIs to target leukocyte recruitment to the site of nascent inflammation, rather than relying on damping down the activation of the leukocytes once they have reached their target tissue.

It is envisioned that BSCIs can be used in at least two distinct ways to treat a disease with an inflammatory component. In the first application, described previously (see for example Grainger & Reckless, Biochem Pharmacol 65(2003) 1027-34; WO 05/053702; WO 06/134384; WO 06/016152; GB 07 15068.3), a medicament with a BSCI compound as its only active ingredient is used as a replacement for existing anti-inflammatory medications such as corticosteroids or cyclooxygenase inhibitors, as a result of their superior selectivity for pathological, as opposed to physiological, inflammation and immune system processes.

In the second application, described and claimed herein, BSCIs are co-administered with a second anti-inflammatory medicament, such as a corticosteroid or cycloxygenase inhibitor, so that the latter medicament can be delivered at a lower dose to achieve the same level of efficacy but with a much-improved side-effect profile. This second approach may be particularly useful where administration of a BSCI alone is insufficiently effective (it is likely that acylaminolactam BSCIs, even at high doses, have a less powerful general anti-inflammatory effect than corticosteroids, since acylaminolactam BSCIs primarily affect neutrophil and macrophage recruitment, as well as certain T cell subsets, which have little or no effect on B cells), or where the second anti-inflammatory agent has other beneficial properties not shared by BSCIs (for example, cyclooxygenase inhibitors have useful antinociceptive effects not shared by BSCIs).

There are a number of generic approaches which can be adopted to limit the impact of side-effects during drug design and development. One approach would be to design or identify entirely new compositions that retain the intended beneficial effects of the original agent, but are more specific and have less diverse molecular interactions and pharmacologic impacts. However, this approach has several major drawbacks. Firstly, there is no generally successful method for identifying such compositions, and it may have been difficult, time consuming and costly to identify even the original agent with the side-effects. Secondly, some or all of the side-effects may be a direct or indirect consequence of the same molecular interaction(s) that were responsible for the target beneficial effect (the immunosuppressive consequences of inhibiting leukocyte activation would be an example of such an effect). In these instances it will be almost impossible to retain the profile of beneficial effects independently from the side-effects.

A second approach, which has previously been used successfully elsewhere, is to combine more than one active ingredient into a single composition, the combination having superior properties to either component administered alone, or to the same two ingredients administered to the same individual but at different times.

Two different concepts underlie the success of the combination approach. In one scenario two drugs which have similar effects but differing molecular mechanisms of action are combined, such that the two ingredients show a synergistic impact on the target factor. By using two ingredients acting synergistically it is possible to administer markedly lower doses of each ingredient in order to achieve the same beneficial effect. Provided the side-effects do not also show synergistic increases (which, provided they depend on molecular interactions which differ from the target effect, they likely will not) such a composition will likely give the same beneficial effects with a reduced burden of side-effects. Indeed, even if the two agents show only additive (as opposed to synergistic) effect then a combined composition will still show reduced side-effects for the same degree of beneficial effect (although the benefit of administering them as a single composition rather than as two separate treatments will likely be less marked). There are numerous examples of such compositions, which combine two active ingredients in a single preparation. For example, Plachetka et al (U.S. Pat. No. 5,872,145 dated Feb. 16, 1999) invented a combination of a 5-HT receptor agonist with an analgesic, particularly an NSAID, for the treatment of migraine. Both active ingredients were administered at a dose below those ordinarily considered as the minimum effective dose for each agent separately, such that the combination together achieved a level of efficacy more commonly associated with administering higher doses of the single agents, each of which is accompanied by unwanted side-effects at doses above the minimum effective dose.

In the second scenario, the second active ingredient in the composition is intended to counter the side-effects of the first active ingredient, so that the combination is simultaneously effective and safe. Such compositions are less common, but patented examples have been very successful in certain applications. For example, the use of estrogen-only hormone replacement therapy leads to undesirable uterine hypertrophy, but the combination of estrogen with a progestogen leads to a combined tablet which can be used safely in women with an intact uterus, although the unopposed estrogen is equally effective when used in women with hysterectomy (where the side effects cannot manifest themselves). In this example, it is clearly of considerable clinical advantage to combine the two active ingredients in a single composition because the side-effects are sufficiently severe, and may even (in the case of endometrial cancer) be life-threatening, that the single combined composition precludes the possibility of the patient taking one active ingredient without the other.

Here, we describe pharmaceutical compositions in which two different anti-inflammatory agents, at least one of which is a BSCI, are combined to form a medicament useful for the treatment of a wide range of diseases with an inflammatory component. We demonstrate that such combinations, unexpectedly, show synergistic effects which allow one or both of the active ingredients to be used at markedly lower doses than would otherwise be required. This unexpected synergy results in a combined medication which can achieve the same or higher degree of anti-inflammatory efficacy with less side-effects than the use of either medication alone, or the use of the two medications administered separately to the same patient.

The invention provides the composition and use of a therapeutic agent, comprising at least two active ingredients (as well as any excipient or carrier), where at least one of the active ingredients is a BSCI, and another active ingredient is an anti-inflammatory agent whose use is normally associated with one or more undesirable side-effects.

More specifically, the invention provides the composition and use of a therapeutic agent, comprising at least two active ingredients, where at least one of the active ingredients is a compound of formula (I), below, and another active ingredient is an anti-inflammatory agent whose use is normally associated with one or more undesirable side-effects.

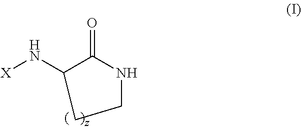

wherein

z is an integer between 1 and 4 inclusive;

X is —CO—Yₖ—(R¹)ₙ or SO₂—Yₖ—(R)ₙ;

k is 0 or 1;

Y is a cycloalkyl or polycyloalkyl group (such as an adamantyl, adamantanemethyl, bicyclooctyl, cyclohexyl, cyclopropyl group);

or is a cycloalkenyl or polycycloalkenyl group;

each R¹ is independently selected from hydrogen or an alkyl, haloalkyl, alkoxy, haloalkoxy, alkenyl, alkynyl or alkylamino radical of 1 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms);

or each R¹ is independently selected from fluoro, chloro, bromo, iodo, hydroxy, oxyalkyl, amino, aminoalkyl or aminodialkyl radical; and

n is any integer from 1 to m, where m is the maximum number of substitutions permissible on the cyclo-group Y (such that n=1 if k=0, such that the R¹ group is bonded directly to the carbonyl or sulfonyl group).

Alternatively R¹ may be selected from a peptido radical, for example having from 1 to 4 peptidic moieties linked together by peptide bonds (for example a peptido radical of 1 to 4 amino acid residues).

Preferably, the compounds of general formula (I) or salts thereof used according to this aspect of the invention will be compounds of general formula (I′)

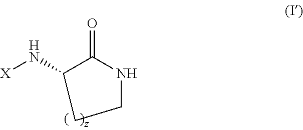

wherein X and z have the same meanings as above.

More preferably, the compound of formula (I) is selected from the following list of compounds:
(S)-3-(2′2′-dimethylpropanoylamino)-caprolactam
(S)-3-(2′2′-dimethylpropanoylamino)-tetrahydropyridin-2-one
(S)-3-(2′2′-dimethylpropanoylamino)-pyrrolidin-2-one
(S)-3-(3′-hydroxy-1′-Adamantanecarbonylamino)-caprolactam
(S)-3-(3′-hydroxy-1′-Adamantanecarbonylamino)-tetrahydropyridin-2-one
(S)-3-(3′-hydroxy-1′-Adamantanecarbonylamino)-pyrrolidin-2-one
(S)-3-(3′-chloro-1′-Adamantanecarbonylamino)-caprolactam
(S)-3-(3′-chloro-1′-Adamantanecarbonylamino)-tetrahydropyridin-2-one
(S)-3-(3′-chloro-1′-Adamantanecarbonylamino)-pyrrolidin-2-one
(S)-3-(3′-fluoro-1′-Adamantanecarbonylamino)-caprolactam
(S)-3-(3′-fluoro-1′-Adamantanecarbonylamino)-tetrahydropyridin-2-one
(S)-3-(3′-fluoro-1′-Adamantanecarbonylamino)-pyrrolidin-2-one

More preferably, the compound of formula (I) will be (S)-3-(2′2′-dimethylpropanoyl amino)-tetrahydropyridin-2-one.

The second active ingredient in the composition is an anti-inflammatory agent whose use is associated with one or more side-effects at the dose typically used to treat an inflammatory condition.

Preferably, the second active ingredient will be a corticosteroid, a cyclooxygenase inhibitor, a non-steroidal anti-inflammatory drug (NSAID) or a TNF inhibitor. For example, the second active ingredient would preferably be selected from the group consisting of dexamethasone, betamethasone, fluticasone, prednisalone, methylpredisolone, cortisone, hydrocortisone, aspirin, indomethacin, sulfasalazine, celecoxib, ruficoxib, piroxicam, tenoxicam, thalidomide, etanercept, infliximab or adalimumab.

More preferably, the second active ingredient will be selected from the group consisting of dexamethasone, betamethasone, fluticasone, prednisalone, methylpredisolone, cortisone and hydrocortisone, since the side-effects of these anti-inflammatory corticosteroids are significantly dose limiting.

It is envisaged that the agent selected as the second active ingredient may also be a BSCI, or have BSCI activity (for example, some BSCIs may have one or more undesirable side-effects and hence qualify under the definition of the second active ingredient in the composition of the invention). In such instances, the second active ingredient will be a structurally distinct BSCI from the first active ingredient. Examples of such combinations envisaged in the present invention would be (S)-3-(2′,2′-dimethylpropanoyl amino)-tetrahydropyridin-2-one combined with yohimban-16-amide, or (S)-3-(2′,2′-dimethylpropanoyl amino)-tetrahydropyridin-2-one combined with (S)-3-(3′-chloro-1′adamantanecarbonylamino)-caprolactam.

It is further envisaged that a composition of the invention may be a fixed dose combination of more than two active ingredients, at least one of which is a BSCI and one of which is an anti-inflammatory medicament associated with one or more undesirable side-effects when used at doses typically used to treat inflammatory conditions. Typically, such a composition will have three active ingredients. Typically, the composition will contain, in addition to the BSCI and the second active ingredient with anti-inflammatory properties associated with one or more undesirable side effects, one further active ingredient designed to ameliorate the symptoms of the particular inflammatory condition to be ameliorated. An example of such a combination envisaged in the present invention would be (S)-3-(2′,2′-dimethylpropanoyl amino)-tetrahydropyridin-2-one combined with fluticasone and salbutamol. In this example, the BSCI has been combined with a well known combination of agents used to treat asthma, such that the dose of the corticosteroid (here, fluticasone) can be reduced while retaining the same degree of anti-inflammatory activity but with a reduced degree of undesirable side-effects (in this example, reduced HPA axis disturbance).

Preferably, the composition of the invention will be administered to the patient as a mixture.

The invention also provides pharmaceutical compositions comprising at least two active ingredients as a mixture, including a compound which is a BSCI, preferably of formula (I), or a pharmaceutically acceptable salt thereof, together with a second anti-inflammatory agent which is usually associated with one or more undesirable side-effects when used at doses typically required for the effective treatment of an inflammatory condition, and at least one pharmaceutically acceptable excipient and/or carrier. For the purposes of this specification, the term ‘mixture’ may optionally include a chemical combination, such as a salt, composed of the two agents according to the invention. Alternatively, the chemical combination may be an ester, or an amide or any similar covalent chemical linkage which allows both components to retain their full pharmaceutical activity.

By pharmaceutically acceptable salt is meant in particular the addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Also within the scope of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217.

The pharmaceutical composition can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical compositions according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

In particular, preferred compositions according to the invention are selected from the following list:
(S)-3-(2′,2′-dimethylpropanoylamino)-tetrahydropyridin-2-one and dexamethasone, betamethasone, fluticasone, prednisalone, methylprednisalone or hydrocortisone;
(S)-3-(2′,2′-dimethylpropanoylamino)-tetrahydropyridin-2-one and aspirin, indomethacin, sulfasalazine, celecoxib or rufecoxib;
(S)-3-(2′,2′-dimethylpropanoylamino)-tetrahydropyridin-2-one and thalidomide, etanercept, infliximab or adalimumab;
(S)-3-(3′-chloro-1′adamantanecarbonylamino)-caprolactam and dexamethasone, betamethasone, fluticasone, prednisalone, methylprednisalone or hydrocortisone;
(S)-3-(3′-chloro-1′adamantanecarbonylamino)-caprolactam and aspirin, indomethacin, sulfasalazine, celecoxib or rufecoxib;
(S)-3-(3′-fluoro-1′adamantanecarbonylamino)-caprolactam and dexamethasone, betamethasone, fluticasone, prednisalone, methylprednisalone or hydrocortisone;
(S)-3-(3′-chloro-1′adamantanecarbonylamino)-tetrahydropyridin-2one and dexamethasone, betamethasone, fluticasone, prednisalone, methylprednisalone or hydrocortisone;
(S)-3-(3′-fluoro-1′adamantanecarbonylamino)-tetrahydropyridin-2one and dexamethasone, betamethasone, fluticasone, prednisalone, methylprednisalone or hydrocortisone;

and any pharmaceutically acceptable salts thereof.

The invention includes compounds, compositions and uses thereof as defined, wherein the compound is in hydrated or solvated form.

It is envisaged that the first active ingredient, with BSCI activity, will be present at a dose similar to or lower than the dose typically used when the agent is administered alone as an anti-inflammatory medicament. For example, if the first active ingredient is (S)-3-(2′2′-dimethylpropanoylamino)-tetrahydropyridin-2-one, such a BSCI would typically be used in the range of 0.1 mg to 250 mg per day, or more typically in the range 1 mg to 50 mg per day, or more typically in the range 20-40 mg per day.

It is envisaged that the second active ingredient, the anti-inflammatory agent associated with one ore more undesirable side-effects when used at doses at doses typically required for the effective treatment of an inflammatory condition, will either: (a) be used at doses lower than the dose typically used when the agent is administered without combination with a BSCI for the treatment of the said condition. For example, hydrocortisone is typically used at a dose of 30 mg per day, via the topical route, for the treatment of psoriasis. A combination of hydrocortisone with a BSCI, according to the present invention, would typically contain hydrocortisone at a dose lower than 30 mg per day, preferably between 0.1 mg and 25 mg, more preferably between 1 mg and 5 mg.

According to this invention, disorders intended to be prevented or treated by the compositions of the invention, or the pharmaceutically acceptable salts thereof or pharmaceutical compositions or medicaments containing them as active ingredients include notably:
autoimmune diseases, for example such as multiple sclerosis, rheumatoid arthritis, Crohn's disease, Grave's disease, mysethenia gravis, lupus erythromatosis, scleroderma, Sjorgren's syndrome, autoimmune type I diabetes;
vascular disorders including stroke, coronary artery diseases, myocardial infarction, unstable angina pectoris, atherosclerosis or vasculitis, e. g., Behçet's syndrome, giant cell arteritis, polymyalgia rheumatica, Wegener's granulomatosis, Churg-Strauss syndrome vasculitis, Henoch-Schönlein purpura and Kawasaki disease;
asthma, allergic rhinitis or chronic occlusive pulmonary disease (COPD);
osteoporosis (low bone mineral density);
tumor growth;
organ transplant rejection and/or delayed graft or organ function, e.g. in renal transplant patients;
psoriasis;
allergies;
Alzheimer's disease, and other idiopathic dementias resulting from neurodegeneration;
Parkinson's disease;
Huntington's disease;
Traumatic brain injury (such as head injuries resulting from a motor vehicle accident), as well as the chronic sequelae (such as impaired memory) resulting from such acute traumatic injuries

Where legally permissible, the invention also provides a method of treatment, amelioration or prophylaxis of the symptoms of an inflammatory disease by the administration to a patient of a therapeutically effective amount of a composition or medicament as claimed herein.

Administration of a medicament according to the invention can be carried out by topical, oral, parenteral route, by intramuscular injection, etc.

The administration dose envisaged for a medicament according to the invention is comprised between 0.1 mg and 10 g depending on the type of active compound used.

The compositions of the invention are readily manufactured using methods which are well known in the art. In particular, the individual active pharmaceutical ingredients may be synthesised by methods well known in the art, and many are commercially available. Except where the two or more active ingredients are chemically combined, the two or more active pharmaceutical ingredients which compose the composition of the invention are then mixed together, preferably as a finely divided powder so that a homogenous mixture is achieved, then added to appropriate pharmaceutical carriers and/or excipients using techniques well known in the art. The mixture, together with any carriers and excipients, is then prepared in a form suitable for administration to a human, for example as a tablet, capsule, liquid suspension or suppository, using methods well established in the art.

Where the composition of the invention includes two or more active pharmaceutical ingredients which are chemically combined, for example as a salt, then the combination is prepared using methods well known in the art. For example, to prepare a salt one of the active ingredients as the free base in an appropriate solvent (such as DMSO or ethanol) is treated with an equimolar amount of the other active ingredient as the free acid, the acid and base then react together to form the salt (plus water). After an appropriate period of time (for example, overnight), the solvent is removed, for instance by use of a vacuum pump, and the solid salt can be used as the composition of the invention. Other methods of counterion exchange are well known in the art, and can be similarly be used to prepare salts of the invention from alternative starting materials, such as the chloride salt of one active ingredient and the sodium salt of the second active ingredient.

Where the composition of the invention includes two or more active pharmaceutical ingredients which are chemically combined, in a single covalently linked compound (for example, an ester linking one active ingredient with a free carboxylate group and a second active ingredient with a free alcohol group), the ester is prepared by methods well known in the art. For example, a mixture of acid and alcohol in an appropriate solvent (such as toluene) may be induced to form an ester by either acid-catalysis or base catalysis depending on the stability of the constituents. Alternatively, an activated form of the acid component can first be prepared (such as an acid chloride or an acid anhydride) which will react with the hydroxylated component directly without the need for catalysis. The general methods for the preparation of such activated acid intermediates, and their subsequent use to form esters are well known in the art.

The following examples are presented in order to illustrate the above procedures and should in no way be considered to limit the scope of the invention.

### Example 1

### Unexpected Synergistic Effects of (S)-3-(adamantylamino)-caprolactam and Dexamethasone in endotoxemia

One composition according to the invention is a mixture composed of (S)-3-(adamantylamino)-caprolactam as the first active ingredient (a well known BSCI; see for example WO 05/053702 and WO 06/018609) and dexamethasone as the second active ingredient, selected such that the combination of the BSCI with the steroid will reduce the dose of steroid required and hence the side-effects associated with chronic, high dose steroid use.

In order to test the impact of combining the ingredients on the anti-inflammatory effect of the composition, which is the primary mode of efficacy of the compositions of the invention, we examined the ability of the combined composition to inhibit leukocyte recruitment and hence systemic TNF-α production in response to a standardised endoxtoxin challenge in vivo, and compared the combination with the two agents administered separately.

Methods

We have used the sub-lethal LPS-induced endotoxemia assay to demonstrate the generalised anti-inflammatory properties in vivo of previously disclosed BSCIs (see, for example, Fox et al. J Med Chem. 2002 Jan. 17; 45(2):360-70; Fox et al. J Med Chem. 2005 Feb. 10; 48(3):867-74; WO 05/053702; WO 06/016152; WO 06/134385; and WO 06/134384). In this assay, mice are given a non-specific pro-inflammatory challenge using bacterial endotoxin (LPS), and the extent of the systemic inflammatory response (measured by serum levels of the central pro-inflammatory cytokine TNF-α, which is essentially absent from the blood under normal conditions, but is rapidly elevated in response to a wide range of inflammatory stimuli). We have selected this model, even though it is not, itself, a particularly close model of any human inflammatory disease condition, because TNF-α is known to be important in very many diseases (including rheumatoid arthritis, autoimmune disorders, Crohn's Disease, atherosclerosis, asthma and many more). Consequently, agents which suppress TNF-α production are already used clinically (e.g. etanercept (Enbrel™) and other anti-TNF-α antibody products, such as infliximab (Remicade™) and adalimumab (Humira™)) to treat a wide range of such diseases. Demonstration of TNF-α suppressive activity in this model is therefore highly predictive of a clinically useful anti-inflammatory effect in a wide range of diseases.

Mice (adult female CD-1 mice in groups of 6) were pretreated with various doses of each compound, either by the subcutaneous route 30 mins prior to LPS challenge, or by the oral route (via gavage) 60 mins prior to LPS. The mice were then challenged with an intraperitoneal injection of 750 μg of bacterial LPS and sacrificed 2 hours later. Serum was prepared from a terminal bleed by cardiac puncture, and the concentration of TNF-α determined by ELISA (R&D Systems). In each experiment, a group of 6 mice receive no LPS to act as a negative control, and a second group receive only LPS (with no candidate inhibitor). The level of TNF-α in serum from these animals which received LPS without drug pre-treatment is arbitrarily set to 100% (and is typically of the order of 6,000 pg/ml, compared with levels of <10 pg/ml among the negative control group, which received no LPS).

Results

In a first series of experiments the concentration of the BSCI (S)-3-(adamantylamino)-caprolactam (‘B’) and of the synthetic corticosteroid Dexamethasone (‘DMX’) required to inhibit LPS-induced TNF-α was determined when the compounds were administered separately. When seeking to determine whether two agents in combination show unexpected synergistic benefits it is important to first perform separate dose-response curves with the two agents to ensure that a sub-maximal dose of each agent is subsequently combined. If, mistakenly, a maximally effective dose of one (or both) compounds were used, such that inflammation were completely suppressed, then it would not be possible to detect any unexpected superior efficacy from the combination.

The dose response curve for DMX by both the sub-cutaneous (triangles) and oral (squares) dosing routes is shown in FIG. 1. The dose response curve for B by the oral route is shown in FIG. 2. It is evident from these graphs that both compounds, when administered separately, are potent anti-inflammatory agents, significantly reducing TNF-α when administered at doses as low as 1 μg per mouse (˜33 μg/kg bodyweight, or equivalent to a 2 mg dose in a 60 kg human). Both compounds are also powerful anti-inflammatory agents, reducing TNF-α in response to an LPS injection by at least 80% at the higher doses tested.

To determine whether the two agents showed synergistic anti-inflammatory effects, we treated groups of mice in the same experimental model with a single oral gavage combining the two agents at doses which, when administered singly, had negligible effect on the TNF-α response. Simultaneous treatment of mice with 0.3 μg per mouse of DMX and 0.1 μg/mouse of B (which, when administered separately cause a minor anti-inflammatory effect which was not statistically significant in every experiment; Table 1) resulted in a reproducible 50-75% reduction in LPS-induced TNF-α levels (Table 1; FIG. 3).

TABLE 1
Synergistic effect of a combined low dose of a BSCI (‘B’) and
dexamethasone (DMX) in the LPS sub-lethal endotoxemia murine
model. Under each treatment condition (all via the oral route) the mean
percentage inhibition of LPS-induced serum TNF-α is reported
(with standard error; SEM) for a group of six mice.
Results from two completely independent experiments are shown.

| | Experiment 1 | | Experiment 2 | |
|---|---|---|---|---|
| | % Inhibition | | % Inhibition | |
| Treatment | Mean | SEM | Mean | SEM |
| DMX (3 × 10⁻⁷ mg) | 19 | 20 | 37 | 8 |
| B (1 × 10⁻⁷ mg) | 15 | 24 | 34 | 9 |
| B (3 × 10⁻⁶ mg) | 34 | 10 | 54 | 6 |
| DMX (3 × 10⁻⁷ mg) & B (1 × 10⁻⁷ mg) | 55 | 11 | 72 | 7 |
| DMX (3 × 10⁻⁷ mg) & B (3 × 10⁻⁶ mg) | 63 | 8 | 94 | 3 |

Similar results were obtained with a higher (but still sub-maximal) dose of B (3 μg/mouse). Once again, in the presence of an ineffective dose of DMX (0.3 μg/mouse), the combination resulted in a substantially greater anti-inflammatory effect than either compound administered separately (Table 1; FIG. 3).

These experiments were repeated twice, with consistent results (Table 1) confirming the reproducible nature of the synergistic effect.

Conclusions

Taken together, these experiments show that the BSCI (S)-3-(adamantylamino)-caprolactam and dexamethasone show unexpected synergistic effects, and that the combination is considerably more potent and powerful as an anti-inflammatory agent in vivo than either compound administered separately, and indeed more powerful and potent than could have been predicted from a simple additive combination of their effects.

### Example 2

### Unexpected Synergistic Effects of (S)-3-(2′,2′-dimethylpropanoylamino)-tetrahydropyridin-2-one and dexamethasone in asthma

In order to examine the impact of combining a BSCI with another anti-inflammatory agent, in this case the corticosteroid dexamethasone, as a mixture on the anti-inflammatory effects in a rat model of asthma, ovalbumin-sensitised animals are treated with (S)-3-(2′,2′-dimethylpropanoylamino)-tetrahydropyridin-2-one, dexamethasone and a mixture of the two agents according to the invention.

Ovalbumin-sensitised rats are selected because they are the most commonly used model of asthma in rodents. In addition, the effect of both dexamethasone and BSCIs in this model has been well characterised (GB 07 15068.3). The extent of leukocyte recruitment into the lung following intratracheal challenge with ovalbumin is used as an indicator of therapeutic efficacy, while beneficial changes in the Th1/Th2 polarisation axis is used to demonstrate the general anti-inflammatory efficacy of the agents. Finally, the suppression of serum growth hormone (GH) levels, a well established side-effect of corticosteroid therapy, is measured to allow comparison of the side effects of the different treatment regimens used.

Methods

Briefly, adult Brown Norway rats (200-300 g body weight; n=10 per group) are sensitised by a single interperitoneal injection of 0.1 mg Ovalbumin on day 0. Each rat then receives an intratracheal challenge with a solution of 1% ovalbumin (w/v) on day 8, and with 2% ovalbumin (w/v) on days 15, 18 and 21. The animals are then sacrificed 3 hours after the final challenge on day 21. Note that ovalbumin (Sigma; purest available grade) can be made endotoxin-free by passage over EndoTrap Red columns (purchased from Cambrex; used in accordance with the manufacturer's instructions), and the endotoxin level confirmed as <5 EU/mg protein using the LAL assay (QCL-1000; Cambrex; performed in accordance with the manufacturer's instructions; 1 mg of standard endotoxin contains ˜900,000 EU/mg). This ensures that the lung inflammation response results from the allergic response to the ovalbumin protein, rather than from unintended LPS stimulation which occurs even with the highest purity grade commercial ovalbumin preparations, and therefore ensures the model more closely represents the underlying molecular pathology of human asthma.

One group of mice (acting as a baseline control) receives no ovalbumin challenges, but are otherwise treated identically. A second group (positive control) receives the challenges but no drug treatment. Further groups are treated identically, but receive daily dosage with: (a) (S)-3-(2′,2′-dimethylpropanoylamino)-tetrahydropyridin-2-one (B′) at a dose of either 0.3 mg/kg or 0.03 mg/kg via oral gavage from day 8 until day 21, with dosage being given 1 hr prior to any subsequent challenge with ovalbumin made on the same day. B′ is administered as a sterile solution in endotoxin-free phosphate buffered saline; or (b) dexamethasone (DMX) at 1 mg/kg or 0.01 mg/kg via the oral route, in an identical treatment schedule to the BSCI; or (c) the same treatment schedule but with a solution containing both 0.01 mg/kg DMX and 0.03 mg/kg B′ as a mixture in accordance with the present invention.

On sacrifice, total lung leukocyate recruitment is assessed by performing a broncheoalveolar lavage (BAL) using 4 lots of 3 ml sterile phosphate-buffered saline introduced through a tracheal cannula. For each animal, the BAL washes are combined, and the total cell population counted (using a haemocytometer).

The spleen is also removed from each mouse and placed in RPMI+10% FCS+antibiotics. The spleens are then each pressed through fine-mesh (100 μm) nylon screens in sterile sieve cups placed in sterile petri dishes to produce single-cell suspensions. The resulting cell suspensions are then centrifuged (328 g; 5 mins) and washed in RPMI+10% FCS+antibiotics, before being resuspended in fresh media and counted using a haemocytometer.

4×10⁶ total splenocytes (excluding RBCs) in total are then cultured (37° C.; 5% CO₂) in RPMI+10% FCS+antibiotics overnight in presence of 2 U/ml (10 ng/ml) murine IL-2 in 4 wells of a 96 well plate (100 μl volume per well/1×10⁶ cells/well) from each mouse. Approximately 24 hrs later, the 4 wells are split into two groups of 2 wells: one group are left untreated, while the second group are stimulated with 500 ng/ml Ionomycin and 50 ng/ml PMA for 4 hours at 37° C. During the last two hours of this incubation 10 μg/ml Brefeldin A (stock 1 mg/ml in EtOH) is added to one well from each set. Brefeldin A blocks protein transport to golgi and therefore allows accumulation of proteins in ER.

The wells without Brefeldin A are incubated for a further 48 hours at 37° C. At the end of the incubation, the cell suspensions are centrifuged (328 g; 5 mins) and the supernatant subjected to ELISA assays (R&D Systems; performed in accordance with the manufacturer's instructions) for murine IL-4 (a marker of Th2 cells) and murine interferon-γ (IFN-γ; a marker of Th1 cells).

The wells with Brefeldin A are stained for intracellular IL-4 and IFN-γ immediately at the end of the four hour incubation as follows: cells stained with anti-CD4-FITC antibody (eBioscience Rat IgG2b, Cat. Code. 11-0041) for 30 mins on ice, then washed in Dulbecco's PBS and fixed with 2% paraformaldehyde (final concentration) in Dulbecco's PBS for 20 mins. After fixation cells are made permeable with Dulbecco's PBS/1% BSA/0.5% saponin (Sigma S7900) for 10 mins at room temperature. The cells from each well are then split into three separate FACS tubes and incubated with:
IFN-γ-PE (eBioscience Rat IgG1, Cat. Code. 12-7311-82, 100 μg); or
Il-4-PE (eBioscience Rat IgG1, Cat. Code. 12-7041-82, 100 μg); or
Isotype controls (a mixture of Rat IgG2b-FITC, eBioscience Cat. Code 11-4031 and Rat IgG1-PE, eBioscience Cat. Code 12-4301)

for 30 mins at room temperature. Cells are then washed (twice with PBS/BSA/saponin and then with PBS/BSA without saponin to allow membrane closure) and resuspended in Dulbecco's PBS ready for flow cytometry analysis.

Cells with specific staining for CD4 on the FITC channel (identifying them as T-helper cells) are analysed for the presence of specific staining for either IL-4 or IFN-γ on the PE channel. The ratio of CD4+ cells staining positive for IFN-γ to CD4+ cells staining positive for IL-4 is then reported as the Th1/Th2 ratio. Untreated Brown Norway rats have a Th1/Th2 ratio of approximately 2.7 in the spleen (that is, approximately 2.7 times more CD4+ cells in the spleen are synthesising INF-γ as IL-4). Following sensitisation and repeated challenge with ovalbumin, the ratio falls to less than 1.5 demonstrating the marked Th2 polarisation which accompanies asthmatic changes in both rodents and humans (a lower Th1/Th2 ratio indicates relative Th2 polarisation, while an increasing Th1/Th2 ratio indicates a relative Th1 polarisation).

Serum is also prepared from a terminal bleed, and levels of GH are measured using a commercially available ELISA (Diagnostic Systems Laboratories, Inc; DSLabs) in accordance with the manufacturer's instructions.

Results

High dose B′ (0.3 mg/kg) and high dose DMX (1 mg/kg) both inhibit leukocyte accumulation in the lung by more than 80%, consistent with the expected clinically beneficial effects of these compounds in asthma (FIG. 4). In marked contrast, neither compound, when administered alone, has a statistically significant effect on leukocyte accumulation in the lung when administered at much lower doses (0.03 mg/kg B′ or 0.01 mg/kg DMX; FIG. 4).

Unexpectedly, however, administration of low dose B′ and DMX as a combination in accordance with the present invention results in a marked, and statistically significant reduction in lung leukocyte recruitment which is comparable in magnitude to the effect seen with either compound alone when administered at doses at least 10-fold higher.

Although lung leukocyte recruitment is considered the more clinically relevant end-point, nevertheless the beneficial systemic effects on the immune system can be observed by examining the “re-balancing” of the Th1/Th2 axis, which is a major effect of BSCI treatment (GB 07 15068.3). DMX, even at the high, dose is significantly less effective at re-balancing the immune system than treatment with B′ (FIG. 5). Even low dose B′ causes a statistically significant Th1 shift in this model, but the combination of B′ and DMX in accordance with the present invention is unexpectedly superior (FIG. 5).

Finally, we examined the effect of the various treatments on levels of growth hormone (GH) is serum prepared from a terminal bleed, as a measure of the extent of the side-effects of the corticosteroid treatment. As expected, DMX (but not B′) significantly suppressed by GH levels (by as much as 80% in the high dose group), consistent with the known effects on the HPA axis in humans. Low dose DMX suppressed GH, but to a considerably lesser extent (approximately 10%). Interestingly, the combination of low dose B′ and DMX in accordance with the invention suppressed GH levels only to a similar level to the low dose DMX alone (FIG. 6).

Conclusions

Taken together, these experiments show that the BSCI (S)-3-(2′,2′-dimethylpropanoylamino)-caprolactam and dexamethasone show unexpected synergistic effects, and that the combination is considerably more potent and powerful as an anti-inflammatory agent in vivo than either compound administered separately, and indeed more powerful and potent than could have been predicted from a simple additive combination of their effects. This synergistic efficacy was seen on both the clinically relevant end-point of lung leukocyte recruitment, and also on the Th1/Th2 re-balancing which typifies BSCI action on the immune system.

In addition, these results demonstrate that co-administration of low doses of BSCI and corticosteroid as a combination according to the invention allows significant efficacy on clinical relevant and anti-inflammatory end-points (comparable to that achieved with much higher doses of either compound administered alone) while avoiding the side-effects (such as, in this case, growth hormone suppression) associated with higher dose corticosteroid use.

Definitions

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range. Hence, for example, the range of 1 to 6 carbon atoms specified in respect of (inter alia) formula I is intended to include all integers between 1 and 6 and all sub-ranges of each combination of upper and lower numbers, whether exemplified explicitly or not.

As used herein, the term “comprising” is to be read as meaning a fixed dose combination of the agents which are stated comprise the composition of the invention, such that the components are mixed together as part of the manufacturing process, forming an essentially homogenous mixture. For the avoidance of doubt, the co-administration of the two agents which comprise the composition of the invention, even if simultaneous, would not constitute a “mixture” as defined herein. However, as noted above, chemical combinations of the components which comprise the mixture (such as a salt) is envisaged, and constitutes a mixture (or two components in a mixture of three or more components) in accordance with this definition.

As used herein, the term “Broad-Spectrum Chemokine Inhibitor” (or “BSCI”) refers to compounds or agents which inhibit leukocyte migration (but not necessarily all, or any, other responses) to a number of different chemokines, acting through different chemokine receptors, simultaneously. Hence the term BSCI has an operational definition: that is, it is defined by an experimental test in which an appropriate leukocyte cell type or cell line (such as the human myelomonocytic cell line THP-1) is induced to migrate in an appropriate assay set-up (such as the ChemoTx™ plates; NeuroProbe) in response to several chemokines (such as MCP-1, MIP-1α, RANTES, IL-8 and SDF-1α), as well as non-chemokine chemoattractants (such as fMLP and C5a) in the presence or absence of an appropriate concentration of the candidate inhibitor. BSCIs are compounds which inhibit leukocyte migration in response to many, or nearly all, of the chemokines tested, but not migration in response to the non-chemokine chemoattractants. The necessary procedure to define a BSCI, including the appropriate controls which are required, are well known in the art (see, for example, Frow E K, Reckless J, Grainger D J. Tools for anti-inflammatory drug design: in vitro models of leukocyte migration. Med Res Rev. (2004) 24(3):276-9; Grainger D J, Reckless J, Fox D J. Broad spectrum chemokine inhibitors related to NR58-3.14.3. Mini Rev Med Chem. (2005) 5(9):825-3). Such a definition includes, but is not limited to, the families (based on compound structures) of peptidic BSCIs (peptide 3; NR58-3.14.3 and related structures), acyl aminoglutarimides (such as NR58,4), yohimban-16-amides and acyl aminolactams. However, the definition also includes other compounds and agents, whether currently known or not, which can be unambiguously be defined as BSCIs through the application of the appropriate tests known in the art.

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

### FIGURES

FIG. 1 shows the dose-response curve for the treatment of LPS-induced sub-lethal endotoxemia in adult female CD-1 mice with dexamethasone (DMX), administered at various doses by either the sub-cuntaneous (triangles) or oral (squares) route. The extent of the anti-inflammatory effect is estimated by measuring the percentage inhibition of LPS-induced serum TNF-α levels. Values represent the mean inhibition for a group of six animals treated identically; error bars are standard errors.

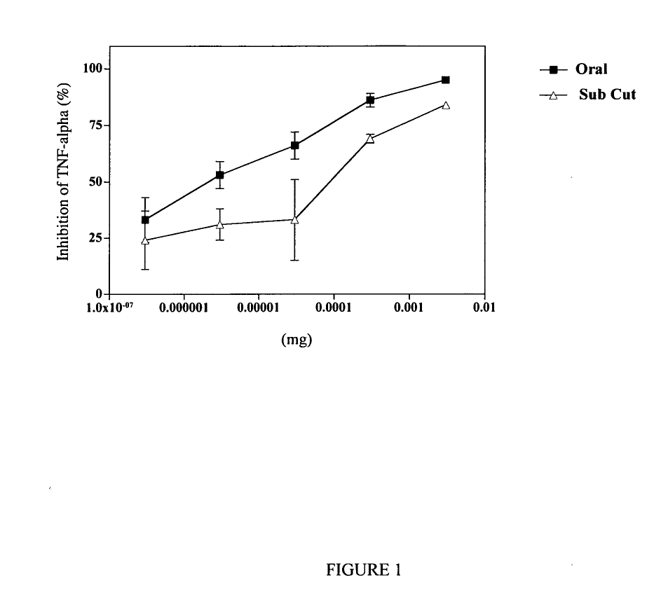

FIG. 2 shows the dose-response curve for the treatment of LPS-induced sub-lethal endotoxemia in adult female CD-1 mice with the BSCI (S)-3-(adamantylamino)-caprolacatm (B), administered at various doses by the oral route. The extent of the anti-inflammatory effect is estimated by measuring the percentage inhibition of LPS-induced serum TNF-α levels. Values represent the mean inhibition for a group of six animals treated identically; error bars are standard errors.

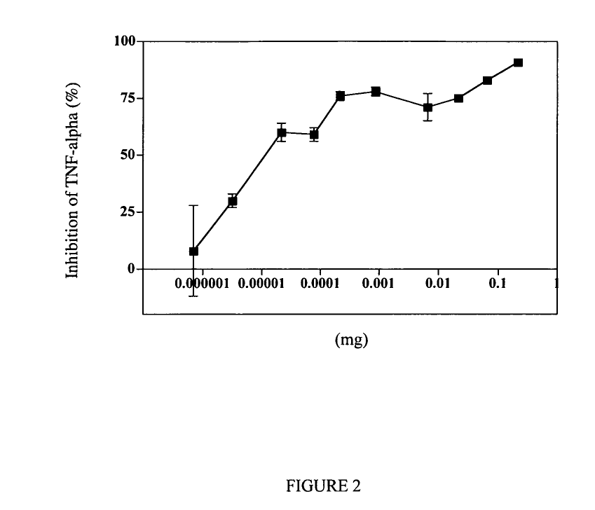

FIG. 3 shows the unexpected synergistic effect of administering a combination of the BSCI (B) and corticosteroid (DMX) as a single oral treatment. Each bar represents the mean inhibition of LPS-induced TNF-a levels in groups of six mice treated identically as shown; error bars are standard errors. The data shown has been pooled from two independent experiments (see Table 1).

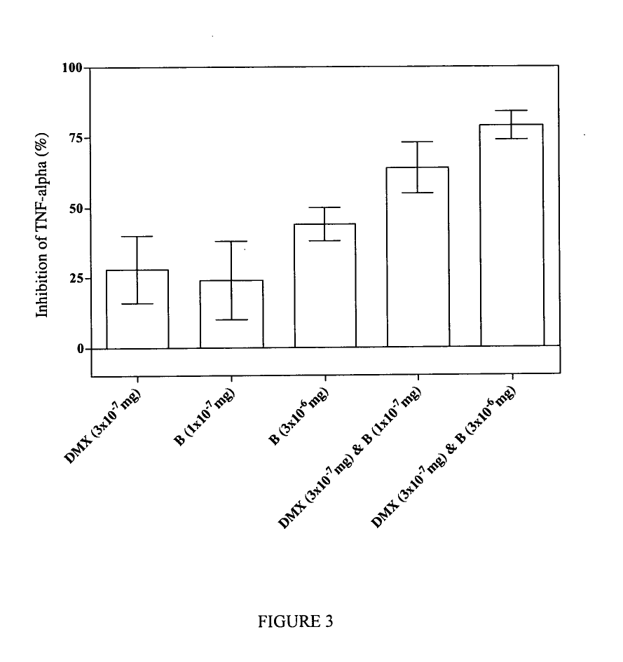

FIG. 4 shows the unexpected synergistic effect of administering a combination of the BSCI (S)-3-(2′,2′-dimethylpropanoylamino)-caprolactam (B′) and corticosteroid (DMX) as a single oral treatment in a rodent model of asthma. The number of leukocytes in the bronchial alveolar lavage (BAL) fluid is shown; bars are mean±standard error for groups of ten rats. While low doses of DMX and B′ administered separately are ineffective, when administered as a combination in accordance with the present invention they have a marked anti-inflammatory effect comparable to that of either compound administered alone but at 10-fold or more higher dose.

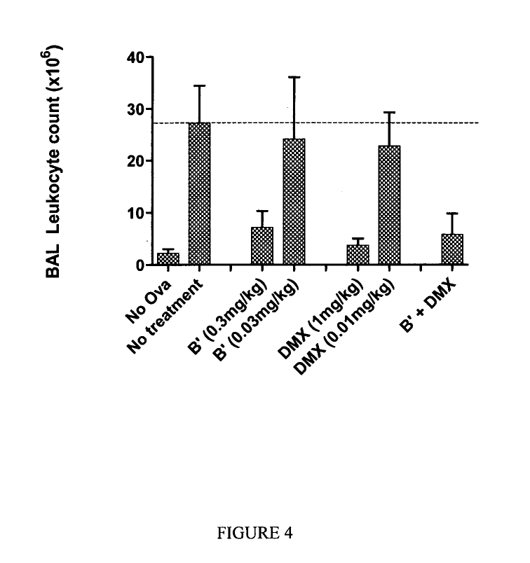

FIG. 5 shows the effect of BSCI (B′) and corticosteroid (DMX) treatment on Th1/Th2 axis polarisation in the same animals as FIG. 4. The bar represents the mean (±standard error) ratio of Th1 cells (CD4⁺/IFN-γ⁺ splenocytes) to Th2 cells (CD4⁺/IL4⁺ splenocytes) in groups of 10 rats treated according to each condition.

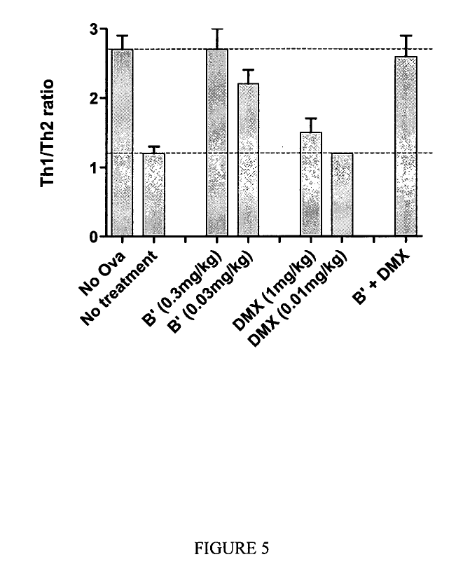

FIG. 6 shows the effect of BSCI (B′) and corticosteroid (DMX) treatment on levels of growth hormone (GH) in serum from the terminal bleed of the same animals shown in FIG. 4. The bar represents the mean (±standard error) concentration of GH in the serum from groups of 10 rats treated according to each condition. Note how the combination of low dose DMX with low dose BSCI only mildly suppresses GH (to a much lesser extent than does high dose DMX) even though this combination according to the invention shows anti-inflammatory effects comparable to the high dose of either compound administered alone.

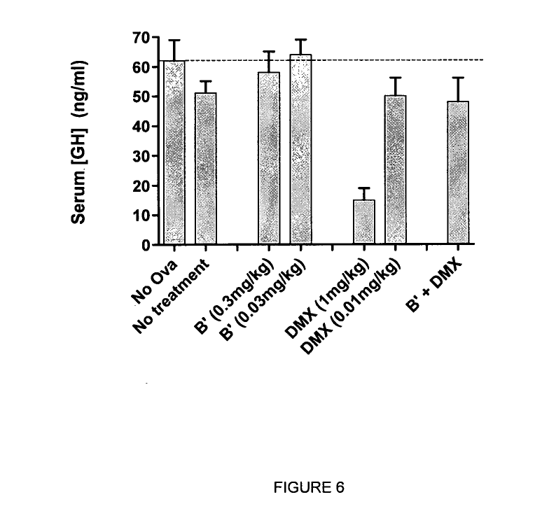

1. Use of a composition, comprising a mixture of at least two active ingredients, or the pharmaceutically acceptable salts thereof, for the manufacture of a medicament intended to treat an inflammatory disorder, where:
(a) the first active ingredient is a Broad-Spectrum Chemokine Inhibitor; and
(b) the second active ingredient is an anti-inflammatory agent associated with one or more side-effects at the dose usually used to treat the inflammatory disorder.

2. A pharmaceutical composition comprising a mixture of at least two active ingredients, or the pharmaceutically acceptable salts thereof, for use as a medicament intended to treat or prevent an inflammatory disorder, where:
(a) the first active ingredient is a Broad-Spectrum Chemokine Inhibitor; and
(b) the second active ingredient is an anti-inflammatory agent associated with one or more side-effects at the dose usually used to treat the inflammatory disorder.

3. The use of a pharmaceutical composition, according to claim 1, wherein the mixture of at least two active ingredients, or the pharmaceutically acceptable salts thereof, is an essentially homogeneous mixture.

4. A pharmaceutical composition, according to claim 2, wherein the mixture of at least two active ingredients, or the pharmaceutically acceptable salts thereof, is an essentially homogeneous mixture.

5. The use of a pharmaceutical composition, according to claim 1, wherein at least one of the active ingredients is present in the mixture at doses lower than the optimal dose of the same active ingredient when administered alone.

6. A pharmaceutical composition, according to claim 2, at least one of the active ingredients is present in the mixture at doses lower than the optimal dose of the same active ingredient when administered alone.

7. The use of a pharmaceutical composition according to claim 1, 3 or 5, where the Broad-Spectrum Chemokine Inhibitor is a compound of formula (I): 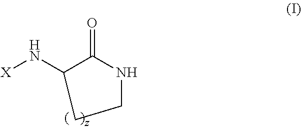
wherein
z is an integer between 1 and 4 inclusive;
X is —CO—Yₖ—(R¹)ₙ or SO₂—Yₖ—(R¹)ₙ;
k is 0 or 1;
Y is a cycloalkyl or polycyloalkyl group (such as an adamantyl, adamantanemethyl, bicyclooctyl, cyclohexyl, cyclopropyl group);
or is a cycloalkenyl or polycycloalkenyl group;
each R¹ is independently selected from hydrogen or an alkyl, haloalkyl, alkoxy, haloalkoxy, alkenyl, alkynyl or alkylamino radical of 1 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms);
or each R¹ is independently selected from fluoro, chloro, bromo, iodo, hydroxy, oxyalkyl, amino, aminoalkyl or aminodialkyl radical; and
n is any integer from 1 to m, where m is the maximum number of substitutions permissible on the cyclo-group Y (such that n=1 if k=0, such that the R¹ group is bonded directly to the carbonyl or sulfonyl group);
alternatively R¹ may be selected from a peptido radical, for example having from 1 to 4 peptidic moieties linked together by peptide bonds (for example a peptido radical of 1 to 4 amino acid residues).
or a pharmaceutically acceptable salt thereof.

8. A pharmaceutical composition according to claim 2, 4 or 6, where the Broad-Spectrum Chemokine Inhibitor is a compound of formula (I): 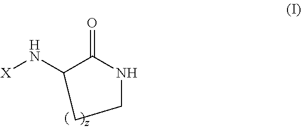
wherein
z is an integer between 1 and 4 inclusive;
X is —CO—Yₖ—(R¹)ₙ or SO₂—Yₖ—(R¹)ₙ;
k is 0 or 1;
Y is a cycloalkyl or polycyloalkyl group (such as an adamantyl, adamantanemethyl, bicyclooctyl, cyclohexyl, cyclopropyl group);
or is a cycloalkenyl or polycycloalkenyl group;
each R¹ is independently selected from hydrogen or an alkyl, haloalkyl, alkoxy, haloalkoxy, alkenyl, alkynyl or alkylamino radical of 1 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms);
or each R¹ is independently selected from fluoro, chloro, bromo, iodo, hydroxy, oxyalkyl, amino, aminoalkyl or aminodialkyl radical; and
n is any integer from 1 to m, where m is the maximum number of substitutions permissible on the cyclo-group Y (such that n=1 if k=0, such that the R¹ group is bonded directly to the carbonyl or sulfonyl group);
alternatively R¹ may be selected from a peptido radical, for example having from 1 to 4 peptidic moieties linked together by peptide bonds (for example a peptido radical of 1 to 4 amino acid residues).
or a pharmaceutically acceptable salt thereof.

9. The use of a pharmaceutical composition according to claim 7, where the compound of formula I has the structure of formula I′: 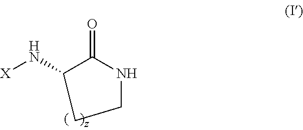
wherein
z is an integer between 1 and 4 inclusive;
X is —CO—Yₖ—(R¹)ₙ or SO₂—Yₖ—(R¹)ₙ;
k is 0 or 1;
Y is a cycloalkyl or polycyloalkyl group (such as an adamantyl, adamantanemethyl, bicyclooctyl, cyclohexyl, cyclopropyl group);
or is a cycloalkenyl or polycycloalkenyl group;
each R¹ is independently selected from hydrogen or an alkyl, haloalkyl, alkoxy, haloalkoxy, alkenyl, alkynyl or alkylamino radical of 1 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms);
or each R¹ is independently selected from fluoro, chloro, bromo, iodo, hydroxy, oxyalkyl, amino, aminoalkyl or aminodialkyl radical; and
n is any integer from 1 to m, where m is the maximum number of substitutions permissible on the cyclo-group Y (such that n=1 if k=0, such that the R¹ group is bonded directly to the carbonyl or sulfonyl group);
alternatively R¹ may be selected from a peptido radical, for example having from 1 to 4 peptidic moieties linked together by peptide bonds (for example a peptido radical of 1 to 4 amino acid residues).
or a pharmaceutically acceptable salt thereof.

10. A pharmaceutical composition according to claim 8, where the compound of formula I has the structure of formula I′: 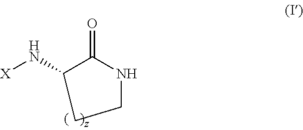
wherein
z is an integer between 1 and 4 inclusive;
X is —CO—Yₖ—(R¹)ₙ or SO₂—Yₖ—(R¹)ₙ;
k is 0 or 1;
Y is a cycloalkyl or polycyloalkyl group (such as an adamantyl, adamantanemethyl, bicyclooctyl, cyclohexyl, cyclopropyl group);
or is a cycloalkenyl or polycycloalkenyl group;
each R¹ is independently selected from hydrogen or an alkyl, haloalkyl, alkoxy, haloalkoxy, alkenyl, alkynyl or alkylamino radical of 1 to 20 carbon atoms (for example of 5 to 20 carbon atoms, of 8 to 20 carbon atoms, of 9 to 20 carbon atoms, of 10 to 18 carbon atoms, of 12 to 18 carbon atoms, of 13 to 18 carbon atoms, of 14 to 18 carbon atoms, of 13 to 17 carbon atoms);
or each R¹ is independently selected from fluoro, chloro, bromo, iodo, hydroxy, oxyalkyl, amino, aminoalkyl or aminodialkyl radical; and
n is any integer from 1 to m, where m is the maximum number of substitutions permissible on the cyclo-group Y (such that n=1 if k=0, such that the R¹ group is bonded directly to the carbonyl or sulfonyl group);
alternatively R¹ may be selected from a peptido radical, for example having from 1 to 4 peptidic moieties linked together by peptide bonds (for example a peptido radical of 1 to 4 amino acid residues).
or a pharmaceutically acceptable salt thereof.

11. The use of a pharmaceutical composition according to claim 9, wherein the compound of structure I′ is selected from the following list:
(S)-3-(2′2′-dimethylpropanoylamino)-caprolactam
(S)-3-(2′2′-dimethylpropanoylamino)-tetrahydropyridin-2-one
(S)-3-(2′2′-dimethylpropanoylamino)-pyrrolidin-2-one
(S)-3-(3′-hydroxy-1′-Adamantanecarbonylamino)-caprolactam
(S)-3-(3′-hydroxy-1′-Adamantanecarbonylamino)-tetrahydropyridin-2-one
(S)-3-(3′-hydroxy-1′-Adamantanecarbonylamino)-pyrrolidin-2-one
(S)-3-(3′-chloro-1′-Adamantanecarbonylamino)-caprolactam
(S)-3-(3′-chloro-1′-Adamantanecarbonylamino)-tetrahydropyridin-2-one
(S)-3-(3′-chloro-1′-Adamantanecarbonylamino)-pyrrolidin-2-one
(S)-3-(3′-fluoro-1′-Adamantanecarbonylamino)-caprolactam
(S)-3-(3′-fluoro-1′-Adamantanecarbonylamino)-tetrahydropyridin-2-one
(S)-3-(3′-fluoro-1′-Adamantanecarbonylamino)-pyrrolidin-2-one
or a pharmaceutically acceptable salt thereof.

12. A pharmaceutical composition according to claim 10, wherein the compound of structure I′ is selected from the following list:
(S)-3-(2′2′-dimethylpropanoylamino)-caprolactam
(S)-3-(2′2′-dimethylpropanoylamino)-tetrahydropyridin-2-one
(S)-3-(2′2′-dimethylpropanoylamino)-pyrrolidin-2-one
(S)-3-(3′-hydroxy-1′-Adamantanecarbonylamino)-caprolactam
(S)-3-(3′-hydroxy-1′-Adamantanecarbonylamino)-tetrahydropyridin-2-one
(S)-3-(3′-hydroxy-1′-Adamantanecarbonylamino)-pyrrolidin-2-one
(S)-3-(3′-chloro-1′-Adamantanecarbonylamino)-caprolactam
(S)-3-(3′-chloro-1′-Adamantanecarbonylamino)-tetrahydropyridin-2-one
(S)-3-(3′-chloro-1′-Adamantanecarbonylamino)-pyrrolidin-2-one
(S)-3-(3′-fluoro-1′-Adamantanecarbonylamino)-caprolactam
(S)-3-(3′-fluoro-1′-Adamantanecarbonylamino)-tetrahydropyridin-2-one
(S)-3-(3′-fluoro-1′-Adamantanecarbonylamino)-pyrrolidin-2-one
or a pharmaceutically acceptable salt thereof.

13. The use of a pharmaceutical composition according to any of claim 1, 5, 9 or 11, wherein the second active ingredient is a natural, semisynthetic or synthetic corticosteroid or corticosteroid mimetic.

14. A pharmaceutical composition according to any of claim 2, 6, 10 or 12, wherein the second active ingredient is a natural, semisynthetic or synthetic corticosteroid or corticosteroid mimetic.

15. The use of a pharmaceutical composition according to claim 13 wherein the corticosteroid is dexamethasone, betamethasone, fluticasone, prednisalone, methylpredisolone, cortisone or hydrocortisone.

16. A pharmaceutical composition according to claim 14, wherein the corticosteroid is dexamethasone, betamethasone, fluticasone, prednisalone, methylpredisolone, cortisone or hydrocortisone.

17. The use of a pharmaceutical composition according to any of claim 1, 5, 9 or 11 wherein the second active ingredient is a non-steroidal anti-inflammatory agent (NSAID).

18. A pharmaceutical composition according to any of claim 2, 6, 10 or 12, wherein the second active ingredient is a non-steroidal anti-inflammatory agent (NSAID).

19. The use of a pharmaceutical composition according to claim 17 where the NSAID is indomethacin, sulfasalzaine, aspirin, celecoxib, ruficoxib, piroxicam or tenoxicam, or an analogue thereof.

20. A pharmaceutical composition according to claim 18 where the NSAID is indomethacin, sulfasalzaine, aspirin, celecoxib, ruficoxib, piroxicam or tenoxicam, or an analogue thereof.

21. The use of a pharmaceutical composition according to any of claim 1, 7, 9 or 11, wherein the second active ingredient is an agent which reduces TNF-α production, bioavailability or biological action.

22. A pharmaceutical composition according to any of claim 2, 8, 10 or 12, wherein the second active ingredient is an agent which reduces TNF-α production, bioavailability or biological action.

23. The use of a pharmaceutical composition according to claim 21, wherein the agent which reduces TNF-α production, bioavailability or biological action is selected from the group consisting of etanercept, infliximab, adalimumab and thalidomide, or an analogue thereof.

24. A pharmaceutical composition according to claim 22, wherein the agent which reduces TNF-α production, bioavailability or biological action is selected from the group consisting of etanercept, infliximab, adalimumab and thalidomide, or an analogue thereof.

25. A pharmaceutical composition, or a use thereof, according to any of the preceeding claims, wherein the active ingredients are (S)-3-(2′2′-dimethylpropanoylamino)-tetrahydropyridin-2-one and a corticosteroid or corticosteroid mimetic, including dexamethasone, betamethasone, fluticasone, cortisone, hydrocortisone, prednisolone or methylprednisolone.

26. A pharmaceutical composition, or a use thereof, according to any of the preceeding claims, wherein one or more further active ingredients are added, which treat one or more symptoms of the disease not directly caused by inflammation.

27. A pharmaceutical composition, or use thereof, according to any of the previous claims wherein the active ingredients, together with any excipients and/or carriers, are formulated as a single tablet.

28. A pharmaceutical composition, or use thereof, according to any of the previous claims wherein two of the active ingredients are chemically combined, in such a way that both retain the activity each possessed when isolated.

29. A pharmaceutical composition, or a use thereof, according to claim 28 wherein two or more of the active ingredients together form a salt.

30. Use of a pharmaceutical composition according to one of claims 1, 5, 7, 9, 11, 13, 15, 17, 19, 21, 23, and 25-29 wherein the inflammatory disorder is selected from the group consisting of autoimmune diseases, vascular disorders, osteoporosis (low bone mineral density), tumor growth, rheumatoid arthritis, multiple sclerosis, organ transplant rejection and/or delayed graft or organ function, psoriasis, eczema, asthma, chronic obstructive pulmonary disease, Crohn's Disease, Irritable Bowel Syndrome or ulcerative colitis.

31. A method of treatment, amelioration or prophylaxis of the symptoms of an inflammatory disease comprising administering a therapeutically effective quantity of the composition according to any of claims 2, 6, 8, 10, 12, 14, 16, 18, 20, 22, 24 and 25-29.
